(19) United States
(12) Patent Application Publication
(10) Pub. No.: US 2010/0292639 A1
(43) Pub. Date: Nov. 18, 2010

(54) CATHETER HAVING SINGLE-LAYER INNER SHAFT

(75) Inventors: Alwin Schwitzer, Buelach (CH); Bettina Surber, Gachnang (CH)

Correspondence Address: BIOTECH BEACH LAW GROUP , PC, 5677 OBERLIN DRIVE, SUITE 204, SAN DIEGO, CA, 92121, US

(73) Assignee: BIOTRONIK VI PATENT AG, Baar (CH)

(21) Appl. No.: 12/774,118

(22) Filed: May 5, 2010

(30) Foreign Application Priority Data
May 14, 2009 (DE) 10 2009 003 114.6

(51) Int. Cl. A61M 25/09; A61M 25/10; B29C 47/00

(52) U.S. Cl. 604/96.01; 604/528; 264/176.1

(57) ABSTRACT

The invention relates to a catheter having a single-layer inner tube, the single-layer inner tube with an inner surface forming a lumen and with an outer surface forming an outer surface of catheter duct, it being possible to arrange one or more guide wires in the lumen of the inner tube, characterized in that the single-layer inner tube is made of a material including: a) 96 to 99.9% by weight of a polymer, a copolymer, and/or a mixture of several polymers and/or copolymers; b) 0 to 1% by weight talc; c) 0 to 1% by weight calcium carbonate; d) 0 to 1% silicon dioxide; e) 0 to 1% by weight waxes; wherein the sum of the weight percentages of constituents b) to e) is at least 0.1% by weight, and no more than 4% by weight, and wherein all weight percentage information relates to the total weight of the single-layer inner tube.

### CROSS REFERENCE TO RELATED APPLICATIONS

This invention claims benefit of priority to Germany patent application serial number DE 10 2009 003 114.6, filed on May 14, 2009; the contents of which is herein incorporated by reference in its entirety.

### FIELD OF THE INVENTION

The invention relates to the field of catheters, particularly catheters for use in angioplasty, and to the field of production methods for components of such catheters.

### BACKGROUND OF THE INVENTION

Catheters, in particular wire-guided catheters, are used widely, primarily in medical applications and procedures. Wire-guided catheters are used particularly in angioplasty and stenting.

A wide variety of wire-guided catheter types are known, for example interventional catheters, balloon catheters, and catheters for the application of self-expanding stents. One common feature of all wire-guided catheters is that they include a tube, referred to as an inner tube, in the lumen of which the guide wires are disposed. Using suitable manipulation of the guide wires, the catheter can be moved in a targeted manner and, for example, be directed to a desired location in the human vascular system. This produces frictional forces between the inside surface of the inner tube and the guide wires. These friction forces can impair the function and safety of the catheter.

Several approaches are known to reduce this friction between the guide wire and inner tube.

For example, DE 696 36 202 describes a catheter arrangement, in which a multilayer inner tube is used. The innermost layer of the inner tube has a surface having a low friction coefficient and is glued to other layers ensuring the necessary stability of the tube.

Another approach is to coat the inside surface of the inner tube with a slip additive, which reduces the friction coefficient of the tube surface.

The known solutions all have in common that they demand complex measures, are cost-intensive, and require several steps or manipulations in the production of the inner tube or to catheter.

### SUMMARY OF THE INVENTION

It is the object of the present invention to mitigate or prevent one or more disadvantages of is the prior art. In particular, catheters are to be provided which have reduced friction between the inner tube and guide wire and can be produced easily, cost-effectively and/or in fewer steps.

The object is achieved by providing a catheter having a single-layer inner tube, wherein the single-layer inner tube with an inner surface forms a lumen and with an outer surface forms an outer surface of catheter duct, with one or more guide wires being arrangeable or arranged in the lumen of the inner tube, characterized in that the single-layer inner tube is made of a material including (a) 96 to 99.9% by weight of a polymer, a copolymer, and/or a mixture of several polymers and/or copolymers; (b) 0 to 1% by weight talc; (c) 0 to 1% by weight calcium carbonate; (d) 0 to 1% by weight silicon dioxide; and (e) 0 to 1% by weight wax or wax mixtures, wherein the sum of the weight percentages of constituents (b) to (e) is at least 0.1% by weight and no more than 4% by weight, and wherein all weight percentage information relates to the total weight of the single-layer inner tube.

By adding one or more constituents (b) to (e) to the primary polymer of the inner tube during or before shaping the inner tube, an inner tube surface is obtained which results in lower friction between the guide wire and tube surface. The effect is based on the fact that the inner tube surface is slightly roughened by the constituents, thereby reducing the contact surface between the wire and inner tube. Since the surface available for contact is thereby reduced, friction as a whole is decreased. This reduction in friction already suffices to ensure a problem-free and safe operation of the catheter, without making the use of additional slip additives or lubricants necessary. Since the constituents can be added to the base polymer of the inner tube during or prior to extrusion, it is possible to produce a single-layer inner tube having the desired properties in just a few steps. In this way, the complex methods and steps for producing multi-layer inner tubes are dispensed with. It also no longer necessary to prepare the surface of the inner tube by the use of additional slip additives, after production of the inner tube.

In principle, the catheter according to the invention encompasses all wire-guided catheter types, such as interventional catheters, catheters for stenting, balloon catheters, or the like, and is not limited to one catheter type. It is essential that the catheter has a single-layer inner tube according to the invention.

With the inside surface, the single-layer inner tube of the catheter forms a lumen in which one or more guide wires of the catheter can be or are arranged. Toward the outside, the surface of the single-layer inner tube forms an outer surface of a catheter duct. The predominant part of the outer surface of the single-layer inner tube is not in direct contact with other layers of the catheter, but the single-layer inner tube instead forms a separate catheter duct, which can be functionally connected to other parts of the catheter, such as other catheter ducts.

The inner tube has a single-layer design. The materials used for producing the inner tube are present in random distribution, in particular in uniform distribution, over the cross-section of the inner tube surface. There are no regions inside the inner tube which differ significantly from other regions of the inner tube in the material composition thereof.

The single-layer inner tube is made of a material including (a) 96 to 99.9% by weight of a polymer, a copolymer and/or a mixture of several polymers and/or copolymers, preferably a polyamide, polyamide copolymers or polyamide-containing copolymers, in particular polyamide 12, polyamide 12/6 and/or polyether block amides (such as PEBAX®), or mixtures thereof; (b) 0 to 1% by weight talc; (c) 0 to 1% by weight calcium carbonate; (d) 0 to 1% by weight silicon dioxide; and (e) 0 to 1% by weight wax or wax mixtures, wherein the sum of the weight percentages of constituents (b) to (e) is at least 0.1% by weight and no more than 4% by weight, and wherein all weight percentage information relates to the total weight of the single-layer inner tube.

The single-layer inner tube includes a material containing a polymer, a copolymer, and/or a mixture of several polymers and/or copolymers. In principle, the polymers or copolymers used can be such which have the necessary stability and flexibility for use in a catheter and can be mixed with the constituents (b) to (e) in the concentrations listed. The person skilled in the art knows such polymers and copolymers, or mixtures thereof, from the prior art. The person skilled in the art will be able to determine the compatibility or miscibility with constituents (b) to (e) and other properties of the polymer in simple routine experiments is without difficulty. Preferably, polymers and/or copolymers are used which include at least one recurring monomer having at least one amide group. In particular, polyamides, polyamide copolymers, or polyamide-containing copolymers can be used. To this end, polyamides having at least 6 carbon atoms per monomer are preferred, such having more than 10 are particularly preferred, and such having 12 carbon atoms are most preferred. In particular, polyamide 12 or polyamide 12 copolymers, such as polyamide 12/6, can be used. It is also possible to use polyether block amides (for example, those available by the trade name of PEBAX®). Furthermore, preferably polymers or copolymers of the group of polyesters, copolyesters and/or polyester elastomers can be used.

Talc, also known as hydrated magnesium silicate, is a mineral having the chemical composition Mg₃Si₄O₁₀(OH)₂. Preferably talc having CAS no. 14807-96-6 is used.

In principle, any calcium carbonate can be used, with precipitated calcium carbonate having CAS no. 72608-12-9 being preferred.

In principle, any silicon dioxide can be used, with precipitated silicon dioxide having CAS no. 7631-86-9 being preferred.

Preferably, stearamide waxes, such as N,N′-ethylene-bis-stearamide (CAS no. 110-30-5) and/or montan waxes (CAS no. 8002-53-7), specifically calcium montanates (CAS no. 68308-22-5) and/or glyceryl montanates (CAS no. 68476-38-0) are used, either individually or as wax mixtures.

The constituents (b) to (e) are present in a concentration each of 0 to 1% by weight, preferably of 0 to 0.5% by weight each, wherein the sum of the weight percentages of constituents (b) to (e) is at least 0.1% by weight, preferably at least 0.5% by weight, and no more than 4% by weight, preferably no more than 2% by weight. All weight percentage information relates to the total weight of the material of the single-layer inner tube. It is not necessary for all constituents (b) to (e) to be present. The material of the single-layer inner tube may include one, two, three and/or four constituents (b) to (e), optionally in different proportions.

The catheter according to the invention may include other components. For example, the catheter may include a dilatable balloon.

In addition to the single-layer inner tube, the catheter according to the invention may include a single-layer or multi-layer outer tube. The outer tube may surround the inner tube at least partially or entirely. Both the outer tube and the single-layer inner tube are separate, independent tubes, which have direct contact with each other over no more than 50% of the outer surface of the inner tube. A lumen may be formed between the inner surface of the outer tube and the outer surface of the inner tube. The lumen can be used, for example, to deliver compounds or substances from the proximal end of the catheter in the direction of the distal end of the catheter. If the catheter according to the invention is a balloon catheter, the lumen between the outer and inner tubes can be used to inflate the dilatable balloon with a fluid.

The present invention also relates to a method for producing a single-layer inner tube for a catheter according to the invention, characterized in that (a) at least two precursors are used, wherein the first precursor is a polymer or copolymer, preferably a polyamide, polyamide copolymer, or polyamide-containing copolymer, and the second precursor includes at least one of the constituents of talc, calcium carbonate, silicon dioxide, and wax, or wax mixtures; (b) both precursors are mixed with each other, and (c) the resulting material mixture is used to produce the inner tube.

The mixing of the two precursors with each other, and the method for producing the inner tube, can also be done simultaneously in a joint step. Similar to the coloring of thermoplastic resins, the two precursors can be mixed with each other and melted only when the inner tube is being produced.

The person skilled in the art knows methods for producing an inner tube starting from a polymer-containing material. The single-layer inner tube according to the invention is preferably produced using an extrusion method. Other suitable methods are injection molding and blown film extrusion, for example.

The first precursor includes a polymer, a copolymer, and/or a mixture of several polymers and/or copolymers. In principle, the polymers or copolymers used in the method according to the invention can be such which have the necessary stability and flexibility for use in a catheter and can be mixed with the constituents (b) to (e) in the concentrations listed. The person skilled in the art knows such polymers and copolymers, or mixtures thereof, from the prior art. The person skilled in the art will be able to determine the compatibility or miscibility with constituents (b) to (e) and other properties of the polymer in simple routine experiments without difficulty. Preferably, polymers and/or copolymers are used which include at least one recurring monomer having at least one amide group. In particular, polyamides, polyamide copolymers, or polyamide-containing copolymers can be used. To this end, polyamides having at least 6 carbon atoms per monomer are preferred, such having more than 10 are particularly preferred, and such having 12 carbon atoms are most preferred. In particular, polyamide 12 or polyamide 12 copolymers, such as polyamide 12/6, can be used. It is also possible to use polyether block amides (for example, those available by the trade name of PEBAX®). Furthermore, preferably polymers or copolymers of the group of polyesters, copolyesters and/or polyester elastomers can be used.

The present invention also relates to a catheter including a single-layer inner tube, produced according to any one of the above methods according to the invention. To this end, the catheters according to the invention can be produced by employing a conventional manufacturing method, wherein the existing inner tube is replaced with a single-layer inner tube produced according to one of the methods according to the invention mentioned above.

### DESCRIPTION OF THE DRAWINGS

The invention is described based on the attached drawings.

FIG. 1 shows an exemplary embodiment of an application device according to the invention as a balloon catheter.

FIG. 2 shows a cross-section of the exemplary embodiment of FIG. 1.

### DETAILED DESCRIPTION OF THE INVENTION

The invention will be explained in more detail hereinafter based on the exemplary embodiments.

### Example 1

### Production of a Single-Layer Inner Tube

### Method 1

The granules of constituents a) and b) are mixed as follows. 97% by weight polyamide 12, Grilamid L25, EMS-GRIVORY is mixed with 3% by weight of the master batch Grilon C MB 7361 FS, EMS-GRIVORY, including the constituents b) to e) in the following concentrations:

b): 0-10% talc
c): 0-10% calcium carbonate
d): 0-10% silicon dioxide
e): 0-10% wax mixtures in
f): 60-99% of a PA612 copolyamides, homogenized, and dried. The mixture is extruded on a commercially available profile extrusion system (L/D 24-28), preferably on a profile extrusion system with D<=20 mm, having a suitable die/mandrel geometry, possibly with the aid of support air, into an inner tube, calibrated to the desired diameters, preferably over a vacuum calibration tank, and cooled in a water bath. The dimensions and/or wall thicknesses are controlled online. In addition, the extruded inner tubes can be subjected to an online visual inspection for imperfections. The sections of the continuous inner tube produced in this way that are to deemed to be OK are cut out online and packaged in a dust-free manner.

### Method 2

The granules of constituents a) and b) are mixed as follows. 96% by weight polyamide 12, Grilamid L25, EMS-GRIVORY is mixed with 4% by weight of the master batch Grilon C MB 8361 FS, EMS-GRIVORY, including the constituents b) to e) in the following concentrations:

b): 0-10% talc
c): 0-10% calcium carbonate
d): 0-10% silicon dioxide
e): 0-10% wax mixtures in
f): 60-99% of a PA612 copolyamides,
homogenized, and dried. The mixture is extruded on a commercially available profile extrusion system (L/D 24-28), preferably on a profile extrusion system with D<=20 mm, having a suitable die/mandrel geometry to a wire to set the inside diameter, calibrated to the desired outside diameter, preferably over a vacuum calibration tank, and cooled in a water bath. The dimensions and/or wall thicknesses are controlled online. In addition, the extrudates can be subjected to an online visual inspection for imperfections. The sections of the continuous extrudate produced in this way that are deemed to be OK are cut out online. In a last step, the wire sections are pulled out of the extrudate sections in order to obtain the inner tubes. These are packaged in a dust-free manner.

### Example 2

### Description of an Embodiment According to the Invention as a Balloon Catheter

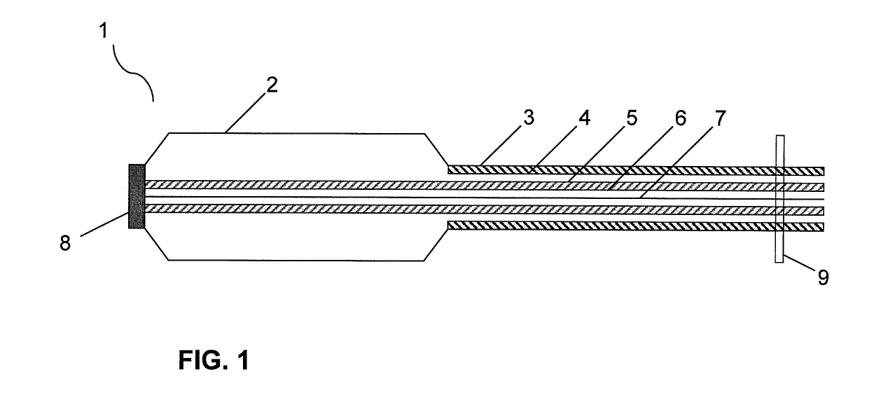

FIG. 1 shows a balloon catheter 1 having a single-layer inner tube 5, to which a distal end of an expandable balloon 2 is fastened, which in a non-expanded, deflated state rests at least partially against an outer surface of the inner tube 5. On the outside of the balloon 2, the balloon catheter 1 may include a stent, which after insertion into a vessel is expanded by inflating the balloon 2 with a fluid and thereby pressed against a vessel wall at the intended site.

In addition to a single-layer inner tube 5 and the balloon 2, the balloon catheter 1 also includes an outer tube 3, which extends at least to a proximal end of the balloon 2 and is connected thereto in a fluid-tight manner. Between the inner tube 5 and the outer tube 3 of the catheter 1 typically a fluid line 4 is provided, which extends in the longitudinal direction of the catheter 1 from the proximal end thereof to the inside of the balloon 2 and which is obtained, for example, in that a lumen is created between the outer tube 3 and the inner tube 5 in which the outer tube 3 has an inside diameter that is larger than the outside diameter of the inner tube 5.

On the inside of the single-layer inner tube 5, a hollow space enclosed by the inner tube 5 and extending in the longitudinal direction of the inner tube 5 is provided as the lumen 6. This lumen 6 is used to receive a guide wire 7. The catheter 1 and guide wire 7 are then designed, for example, such that the guide wire 7 can exit at the distal tip 8 of the catheter 1 and can be controlled from the proximal end thereof. The guide wire 7, for example, is deflected using control means such that it can be easily introduced even into branching blood vessels. Due to the properties of the single-layer inner tube 5, the friction between the inner surface of the single-layer inner tube 5 and guide wire 7 is reduced such that problem-free and reliable operation of the catheter 1 is ensured. The balloon catheter 1 can be advanced along the guide wire 7.

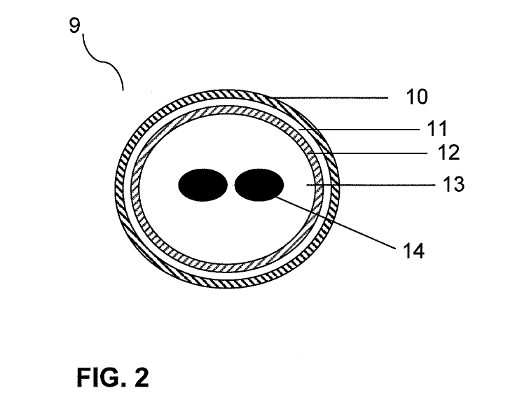

At the distal end, the balloon catheter 1 includes the expandable balloon 2 mentioned above. During insertion of the balloon catheter 1, the balloon 2 is compressed and rests closely against the single-layer inner tube 5 of the catheter 1. By inflating the balloon 2 with a fluid, it can be expanded. This expansion of the balloon 2 is carried out as soon as the balloon 2 has been guided to the intended position. Finally, FIG. 1 shows the plane of a cross-section 9 of the balloon catheter 1, wherein details of the cross-section 9 are illustrated in FIG. 2.

FIG. 2 shows a cross-section 9 of the balloon catheter 1 from FIG. 1 including the tube arrangement. It is apparent how the guide wire 14 is disposed in the lumen 13 of the single-layer inner tube 12, with the single-layer inner tube 12 being surrounded by an outer tube 10. An annular fluid line 11, which surrounds the single-layer inner tube 12 and by which the balloon can be inflated with fluid, is present between the single-layer inner tube 12 and outer tube 10. It is apparent from FIG. 2 that on the catheter according to the invention the single-layer inner tube 12 is designed as a separate tube, which is substantially independent of a possible present outer tube 10.

It will be apparent to those skilled in the art that numerous modifications and variations of the described examples and embodiments are possible in light of the above teaching. The disclosed examples and embodiments are presented for purposes of illustration only. Therefore, it is the intent to cover all such modifications and alternate embodiments as may come within the true scope of this invention.

What is claimed is:

1. A catheter having a single-layer inner tube, the single-layer inner tube with an inner surface forming a lumen and with an outer surface forming an outer surface of a catheter duct, it being possible to arrange one or more guide wires in the lumen of the inner tube, wherein the single-layer inner tube is made of a material comprising:
a) 96 to 99.9% by weight of a polymer, a copolymer, and/or a mixture of several polymers and/or copolymers;
b) 0 to 1% by weight talc;
c) 0 to 1% by weight calcium carbonate;
d) 0 to 1% by weight silicon dioxide;
e) 0 to 1% by weight waxes,
wherein the sum of the weight percentages of constituents b) to e) is at least 0.1% by weight and no more than 4% by weight, and wherein all weight percentage information relates to the total weight of the single-layer inner tube.

2. The catheter according to claim 1, wherein the catheter comprises an outer tube, which at least partially surrounds the single-layer inner tube.

3. The catheter according to claim 1, wherein a lumen is formed between the outer and inner tubes.

4. The catheter according to claim 1, wherein the catheter comprises a dilatable balloon.

5. The catheter according to claim 1, wherein the single-layer inner tube is made of a material comprising at least two of the constituents b) to e).

6. The catheter according to claim 1, wherein the single-layer inner tube is made of a material comprising:
a) 96 to 99.9% by weight of a polymer, a copolymer, and/or a mixture of several polymers and/or copolymers;
b) 0 to 0.5% by weight talc;
c) 0 to 0.5% by weight calcium carbonate;
d) 0 to 0.5% by weight silicon dioxide;
e) 0 to 0.5% by weight waxes,
wherein the sum of the weight percentages of constituents b) to e) is at least 0.1% by weight and no more than 2% by weight, and wherein all weight percentage information relates to the total weight of the single-layer inner tube.

7. The catheter according to claim 1, wherein the sum of the weight percentages of constituents b) to e) is at least 0.5% by weight and no more than 2% by weight, and wherein all weight percentage information relates to the total weight of the single-layer inner tube.

8. A method for producing a single-layer inner tube for a catheter according to claim 1, wherein:
a) at least two precursors are used, wherein the first precursor is a polymer or copolymer, preferably a polyamide, polyamide copolymer, or polyamide-containing copolymer, and the second precursor comprises is at least one of the constituents b) to e);
b) both precursors are mixed with each other, and
c) the resulting material mixture is used to shape the inner tube.

9. A method for producing a single-layer inner tube for a catheter according to claim 1, characterized in that the single-layer inner tube is shaped by way of an extrusion method.

10. A catheter comprising a single-layer inner tube, produced by way of a method according to claim 8.
